(19) United States
(12) Patent Application Publication
(10) Pub. No.: US 2016/0220274 A1
(43) Pub. Date: Aug. 4, 2016

(54) QUICK-RELEASE SYSTEMS FOR EXTRASKELETAL FIXATION

(71) Applicants: Charles H. Lang, JR., Oxnard, CA (US); David J. Mishelevich, Playa delRey, CA (US); Kevin Scott, Huntington Beach, CA (US)

(72) Inventors: Charles H. Lang, JR., Oxnard, CA (US); David J. Mishelevich, Playa delRey, CA (US); Kevin Scott, Huntington Beach, CA (US)

(21) Appl. No.: 15/081,901

(22) Filed: Mar. 27, 2016

Related U.S. Application Data
(63) Continuation-in-part of application No. 14/454,703, filed on Aug. 7, 2014, now abandoned.
(60) Provisional application No. 61/863,425, filed on Aug. 7, 2013.

(51) Int. Cl. A61B 17/64

(52) U.S. Cl. CPC A61B 17/645; A61B 17/6483; A61B 2017/606

(57) ABSTRACT

External fixation assemblies offer good benefits in relevant orthopedic procedures but would be used more efficiently if the quick-release fasteners could be used so no tools would be required. This invention covers various embodiments of quarter-turn, quick-release fasteners, various shapes and surfaces of connecting rods and bone pins or other clamped objects to facilitate use of external fixation assemblies. Included are quick-release fasteners constructed from programmable magnets. Connecting rods can have fastening positions in the form of threaded or non-threaded holes. Both holes and connecting rods and bone pins can be of different shapes, for example, round, square, oval, pentagonal, hexagonal, octagonal, or any arbitrary shape. In both adding holes and non-circular shapes, rotation is eliminated allowing better fixation, and, in the case of holes or dimpled or out-dented surfaces, longitudinal motion along the rod or pin as well.

### CROSS REFERENCE TO RELATED APPLICATIONS

This patent application is a Continuation-in-Part that claims priority to patent application Ser. No. 14/454,703 filed Aug. 7, 2014 that in turn claims priority to Provisional Patent Application No. 61/863,425 filed Aug. 7, 2013, both entitled “QUICK-RELEASE FASTENERS FOR EXTRASKELETAL FIXATION.”

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for orthopedic external fixation to support bones in appropriate relative positions to promote healing.

### BACKGROUND OF THE INVENTION

External fixation varies widely comprising many different types of apparatus. Typically bone pins are inserted through the soft tissue into the bone fragments; devices are then affixed to the pins and serve to connect the bone fragments in such a way as to maintain correct anatomic position during the healing process. As is often the case, placement of the bone pins must be carefully selected to avoid damaging structures such as blood vessels, nerves, tendons, etc. Additionally consideration must be given to the structural integrity of the bone stock in combination with geometric stability considerations of the final construct.

When external fixation devices are used, it is very important that the connecting rods and bone pins are securely held in place by the clamps or attachment blocks. Nonunion of the bone or other structural damage can occur if a connecting rod or bone pin were to become detached or loose while the external fixation device is in use, which could require revision surgery. Many of the prior art external fixation clamps or attachment blocks are secured to the connecting rod or bone pin with a nut and bolt system extending through the collar and center of the clamp. When the screw is tightened, it engages with the connecting rod or bone pin locking them in place. While these clamps are generally effective, the screw must be very tight to adequately secure the connecting rods and bone pins. This frequently makes the clamp with a nut and bolt system very difficult in small spaces and awkward patient positioning. The surgeon may also miss or not completely tighten all bolts. The nut and bolt system is also very difficult to remove or adjust during and after surgical procedures. Often times the surgeon is required to frequently adjust the external fixation device many times during a procedure in order to achieve the optimal bone alignment.

Current connecting rods have smooth surfaces and round shapes. Thus unless the clamps or attachment blocks are fastened tightly they can rotate and lose the desired positioning. Benefits would be achieved if there were receptacles for the fasteners so once positioned the clamp or attachment block could not rotate or move longitudinally. Alternative rod shapes can be helpful as well to aid in the fixation so the clamp or attachment block will not rotate around the connecting rod. Dye (Donald W. Dye, “Orthopedic Instrument with Quarter-Turn Disconnect Mechanism,” U.S. Pat. No. 5,496,323, Mar. 5, 1996) teaches a mechanism providing for a female socket on a cutting instrument and a male plug on the cutting apparatus. While this is an orthopedic application it is not related to external fixation or similar application.

Because of the difficulties associated with using conventional bolts, it would be of benefit to have a quick-release fastener that can be easily engaged or disengaged.

### SUMMARY OF THE INVENTION

The present invention relates to spring-loaded fasteners for connecting a first component to a second component requiring only one quarter turn to switch between a fastening/locking position and a release position.

The present invention, aims to provide greater speed, security, visualization and placement flexibility in a more compact construct than is presently available. It comprises the placement of fixation elements on either side of a fracture. These fixation elements are commonly referred to as pins, whereby one or more pins are screwed into a bone. One or more pins are located on either side of a fracture and connected to a bar via a clamp and/or a clamping system. The present invention relates to connecting rods and bone pins with one or more bars in order to fixate a fracture. Such bars may also be connected to other bars or structures, if needed. In connecting pins to rods, it is advantageous to have mobility in a clamping system to allow for ease of placement and/or post placement manipulation. The present invention also requires no tools to utilize, although use of a tool to engage or release the fastener is not precluded. Embodiments of the device include the capability of the objects clamped to pivot and rotate to many different angles.

### BRIEF DESCRIPTION OF THE DRAWINGS

FIG. 1 shows a diagram of quarter-turn fastener.

FIG. 2 illustrates an extraskeletal fixation device using quarter-turn fasteners.

FIG. 3 shows quarter-turn fasteners including a spring mechanism using a wave washer with FIG. 3A showing the position with the fastener engaged and FIG. 3B the position with the fastener unengaged.

FIG. 4 demonstrates an alternative twist means for a quarter-turn fastener with FIGS. 4A-C showing alternative handle configurations.

FIG. 5 illustrates a frame rod with blind holes for receiving the rod end of a quarter-turn fastener.

FIGS. 6A-6F show alternative cross sections of circular and non-circular connecting rods.

FIG. 7 shows a perspective view of a dimpled connecting rod or bone pin.

FIG. 8 demonstrates a cam-clamp fastener.

FIGS. 9A and B show a dual-sphere locking device with quarter-turn clamps with FIG. 9A showing the internal view and FIG. 9B showing the external view.

FIGS. 10A and B show a dual-sphere device with a rigid lock with FIG. 10A showing the external view and FIG. 10B showing the internal view.

FIG. 11 illustrates a quarter-turn locking mechanism.

FIG. 12 shows a ten-point connecting rod.

FIGS. 13A and B show a parallel clamp with FIG. 13A showing the external view and FIG. 13B the internal view.

FIGS. 14A and B illustrate alternative parallel clamps mechanism, FIG. 14A with octagonal and oval clamping openings and FIG. 14B with two types of stellate clamping openings.

FIG. 15 illustrates a pinch-clamp assembly.

FIG. 16 shows a side, external view of the pinch-clamp assembly of FIG. 15.

FIG. 17 illustrates clamp using twist-release programmable magnets.

FIG. 18A shows the programmed maxel pattern for a twist-release configuration and FIG. 18B illustrates a sample maxel dot pattern.

### DETAILED DESCRIPTION OF THE INVENTION

The invention incorporates a quarter-turn quick-release fastener-based clamp and release mechanism into extraskeletal orthopedic frame assemblies. The quarter-turn mechanism is applicable whether the frame is assembled prior to surgery (the usual practice) or during surgery. As opposed to the quarter-turn fastener, prior art implementations use a conventional screw inserted into a threaded hole. Thus a tool, usually a wrench, is employed to tighten the end of the screw against the connecting rod or the bone pin. This is both inconvenient and requires the operator to determine him or herself how much to tighten the fastener. A quick-release fastener is one that may be engaged or disengaged without the use of a tool.

An articulation clamp consists of a plurality of adjustable jaws for the purpose of fixating the relative position of connecting rods and/or bone pins of an external bone fixator. Projecting elements of the attachment blocks are the bone pins going into the bone and the connecting rods going between the attachment blocks. The clamps or attachment blocks can be square, rectangular, spherical, or combination shapes. Passages are eccentrically arranged through the attachment blocks to accept the bone pin or connecting rod. In one embodiment, Mullaney (Michael W. Mullaney, “Method and Clamping Apparatus for External Fixation and Stabilization,” U.S. Pat. No. 8,241,285, Sep. 14, 2012), commercialized in the ExtraOrtho XO series, each of the adjustable jaws consists of a pair of hemispherical jaw elements contained within a spherical housing. A passage is eccentrically arranged through the jaw elements to accept the bone pin or connecting rod. Another ExtraOrtho embodiment, involving clamps and links, is Miller (Stephen T. Miller, “Clamping Assembly with Links,” US 2012/0289959, filed Nov. 4, 2011). Neither of these embodiments uses quarter-turn release mechanisms. Hibner and Avimukta (Hibner, J. A. and Avimukta, K., “Biopsy Cannula Adjustable Depth Stop, US 2009/0163830, filed as Ser. No. 12/368,317 on Feb. 10, 2009) describe a rotating guide in the biopsy device with quarter-turn locking, but this application is not related to extraskeletal or other bone fixation.

Alleyne and Nonaka (Alleyne, N and M Nonaka, “Bone Fixation Apparatus,” US 2008/0058811, filed at Ser. No. 11/935,990 on Nov. 6, 2007) describe a bone fixation device for spinal stabilization in which a cap is positioned on a receiver with securing them by rotating the cap around one quarter turn. This apparatus is for purpose of containing a threaded bone screw and not either for construction of a frame for extraskeletal fixation or including a quarter-turn fastener where a pin in the turned shaft engages a track in a clamp. Metz-Stavenhagen (U.S. Pat. No. 7,235,075) does specify a quarter-turn fastener and the quarter-turn fastener gripping a rod but does not disclose clamps, cam clamp or connection blocks. The mechanisms only deal with pulling a shaft into a holder, not pushing, do not have a shaft, and require a tool to use. Metz-Stavenhagen is limited to spinal applications and connection blocks required to construct frames for extraskeletal fixation are not specified by Metz-Stavenhagen and neither are bone pins or non-circular connection rods or connecting rods with holes in them to received quarter-turn fastener shafts or where the connection rods have dimples or out-dents. Chin et al. (US 2010/0063552) describe a system for extraskeletal fixation to assemble clamped objects using quarter-turn fasteners with a feature riding in a curved track. The device is used for spinal fixation rather than for assembly of extraskeletal frames. In one primary embodiment, the rotating cap is specifically set up to receive a hexagonal tool and in another primary embodiment, a specific insertion tool is described.

Kuslich (U.S. Pat. No. 5,591,235) describes a clamped object that has a surface that is not necessarily smooth and where the cross section is not round. It is used in implantable systems and not applicable to systems for extraskeletal fixation.

Beside the frame elements, clamps in extraskeletal fixation systems clamp onto connecting rods or bone pins. These are referred to here as clamped objects so they can be referred to as a group. This group can also include other useful elements such as a support that is only clamped at one end.

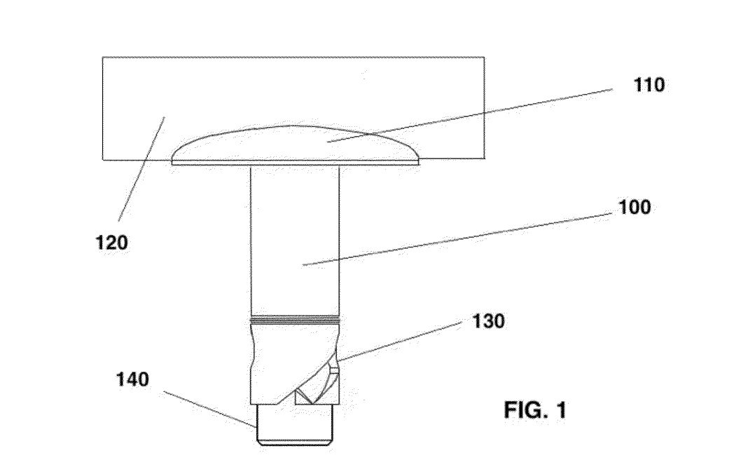

FIG. 1 shows the quarter-turn, quick release fastener. The shaft 100 of the fastener is turned by applying rotational force to fastener head 110 with leverage applied via elongated handle 120 attached to head 110. The quarter-turn mechanism engagement catch 130 grabs a feature such as a pin in the attachment block, not shown, with the fastening into the final fixed position accomplished by twisting handle 110 one quarter turn. Pressure against the rod or pin to be held by the attachment block is through fastener end 140. The handle can be elongated as shown in element 120 or can be shorter or have exposed handles on both sides of fastener head 110. At least one spiral cam slot is on the stud body and a corresponding cam surfaces on the receptacle whereby relative rotation between the stud body and receptacle will cause the cam surface to follow the cam slot or track between the locked and unlocked positions. The fastener may not require a tool to engage or release the fastener. The fastener need not be exactly 90 degrees; embodiments can include rotations in the range of 30 to 150 degrees, but not limited to those end points.

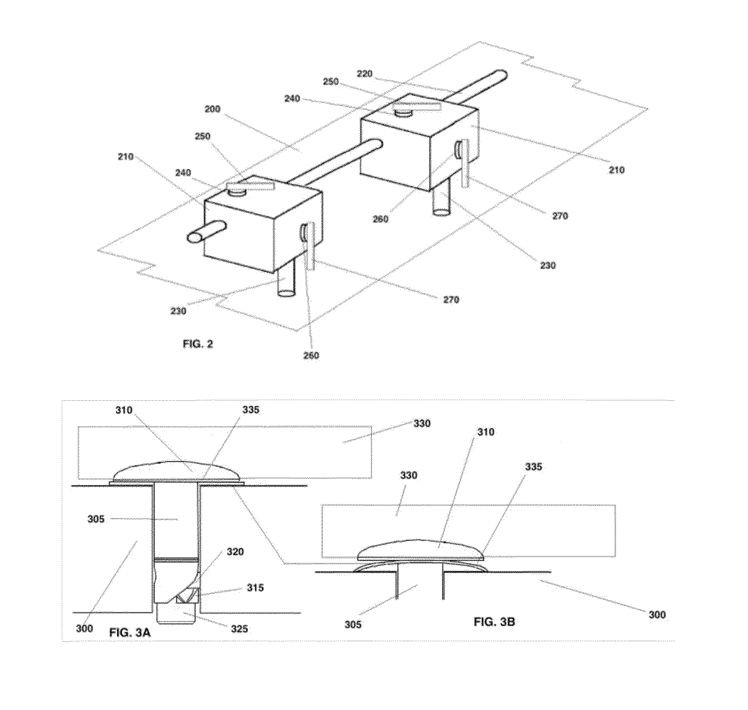

FIG. 2 illustrates a complete assembly associated with fixation of bone segments within body part 200. In this case, two attachment blocks 210 are linked by connecting rod 220 with fastening provided by quarter-turn fasteners 240 turned by elongated handles 250. Bone pins 230 are fastened into the attachment block by quarter-turn fasteners 260 turned by elongated handles 270. More than one such assembly can be used in a given procedure (for example one assembly on each side of a limb being repaired) and there can be cross connecting rods between two assemblies, in which case a cross-connecting rod as well as the connecting rod and a bone pin can be incorporated in a single attachment block or a cross-connecting rod and a connecting rod included in a separate attachment block. The quarter turn system contains a quick release mechanism on the clamp which allows the connecting rods and bone pins to be removed quickly and easily without the use of any tools. In one embodiment, the jaws are attached in a back-to-back fashion through the use of a quarter (¼) turn bolt that acts as a turnbuckle pulling the jaws together when tightened. The quarter turn system will secure connecting rods and bone pins at the optimal compression to prevent loosening and over tightening. Using the quarter turn fastening system plus a flip tab for tightening/locking and loosening requires no tools, thus speeding up the procedure both applying the external fixation system and removing the system.

Competitors with the nut and bolt risk interference from soft tissue, and competitors with two nuts require both to be tightened. Competitive systems therefore unnecessarily prolong surgery, especially when nuts have to be retightened. These efficiencies result in saved operating room time, costs, and surgeon/surgical team effort. Our described invention takes up less space and thus provides for better visualization for patient safety.

FIG. 3 shows the quarter-turn fastener with a wave washer providing tension that allows smooth transition in clamping. The preload of the wave washer or wave spring takes up the slack in the quarter-turn fastener in the disengaged state. In FIG. 3A, quarter-turn fastener 305 fastens attachment block 300 with engagement mechanism 320 engaging-pin feature 315 in attachment block 300. Quarter-turn fastener 300 has a head 310 turned by elongated handle 330 and applies pressure to either a connector rod or a bone pin via rod 325 with a flattened wave washer spring 335 providing tension during turning of the quarter-turn fastener. In FIG. 3B, quarter-turn fastener 305 going through attachment block 300 is shown in its unengaged position with head 310 and handle 330 distanced from the upper surface of attachment block 300 and wave washer 335 in a relaxed state. When quarter-turn fastener 305 is turned it advances into attachment block 300 against tension generated when wave washer 335 is compressed. This offers the operator a smoother transition and can be more ergonomic. In another embodiment, wave washer 335 would be replaced by a coil or other spring with or without a containing a hole concentric with quarter-turn fastener 305 bored partially through attachment block 300. A spring is positioned to be biased as the cam surface follows the spiral cam slots thereby assisting in drawing the two members into tight and locked position. Finally, a retainer is provided for maintaining proper orientation of the fastener when the members are subjected to a shear load so that the primary load on the fastener is a shear load thereby increasing the load therein capacity of the fastener. Embodiments can include cases where the engagement pin is in the receptacle as shown in FIG. 3 or, the engagement pint can be on the shaft of the quarter-turn fastener. The latter case would require an entrance track in the receptacle allowing insertion of said pin down to a portion of the track that is curved to allow fastening engagement by rotating a quarter turn plus a one-way mechanism such as a sprung flap or block that would prevent the pin from being withdrawn back into the entrance track.

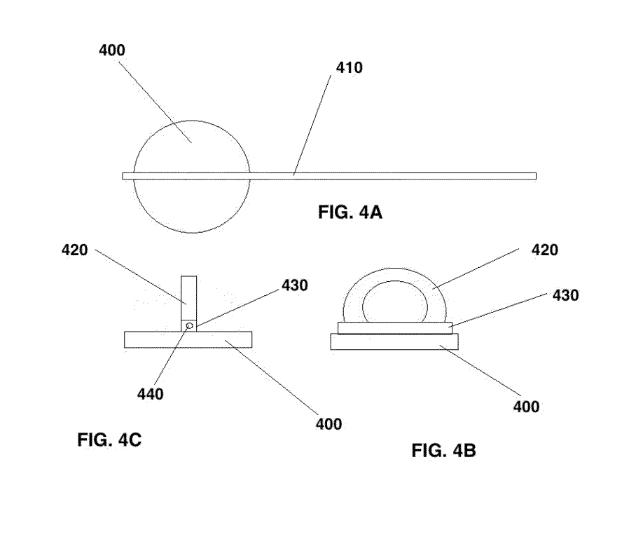

FIG. 4 shows alternative handles for the quarter-turn fastener. FIG. 4 shows a plan view of a quarter-turn fastener with head 400 and elongated handle 410. Another embodiment is shown in FIG. 4B in which quarter-turn fastener head 400 has alternative handle assembly in which twist device 420 is attached to support 430 such that 420 can rotate so it can be flattened and lie parallel to head 400. FIG. 4C illustrates pin 440 contained within support 430 by which pin 400 can rotate around pin 440 and come to a position parallel with quarter-turn head fastener 400 for easy storage or having handle 420 not be sticking out before or after the quarter-turn fastener is engaged. The quarter turn clamp mechanism will have a flip tab that is used to tighten/lock and loosen the clamping device. This tab will turn a quarter turn in one direction for tightening and locking into a color-coded position indicating to the surgeon and staff the clamp is in the locked position, taking the guess work out of knowing if the apparatus is completely locked and secured. The tab will then turn in the opposite direction loosing the clamp, the color-coding will indicate to staff and surgeons that the clamp is in the unlocked position. This quarter turn color-coded tab at the top of the clamp will speed up procedure time and insure proper tightness throughout the procedure.

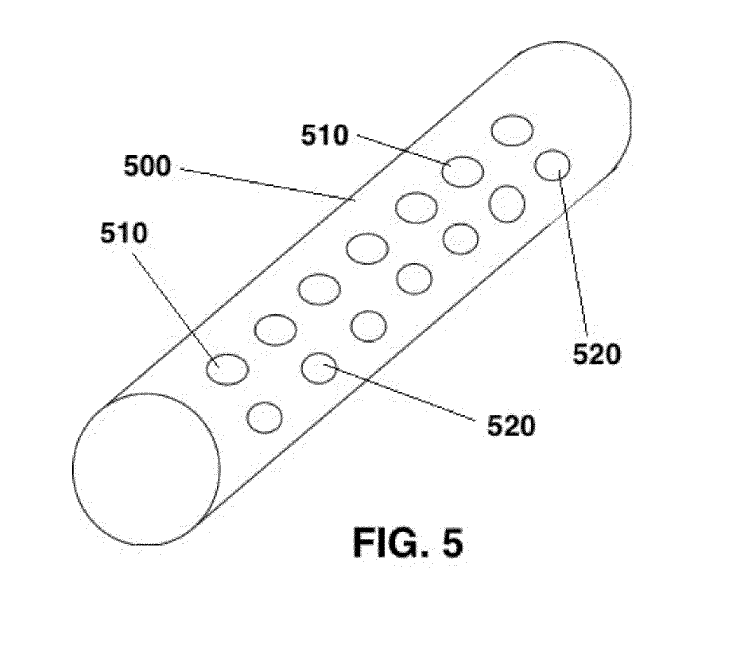

FIG. 5 demonstrates an alternative connecting rod in which the surface is not smooth but has holes partially bored through to receive the pin projecting from the end of the quarter-turn fastener. In FIG. 5, connecting rod 500 has holes 510 on the top and holes 520 on the side. Such holes can also be located at any position around the connecting rod (such as six or eight or other positions as opposed to four positions). The more such positions, the more angles can be accommodated so finer adjustments can be made, particularly if bone pins are already positioned in bone. In addition to preventing rotation, use of the holes would prevent longitudinal movement as well. The holes themselves can be non-threaded or threaded and can be of different shapes such as round, square, rectangular, oval, octagon, hexagonal, octagonal, and arbitrary. A hole may also include a pin mechanism to receive the engaging catch of a quarter-turn fastener or another style of fastener such as a cam-clamp fastener. Employing such surfaces on connecting rods or bone pins can facilitate use of external-fixation assemblies whether quarter-turn clamps are used or not.

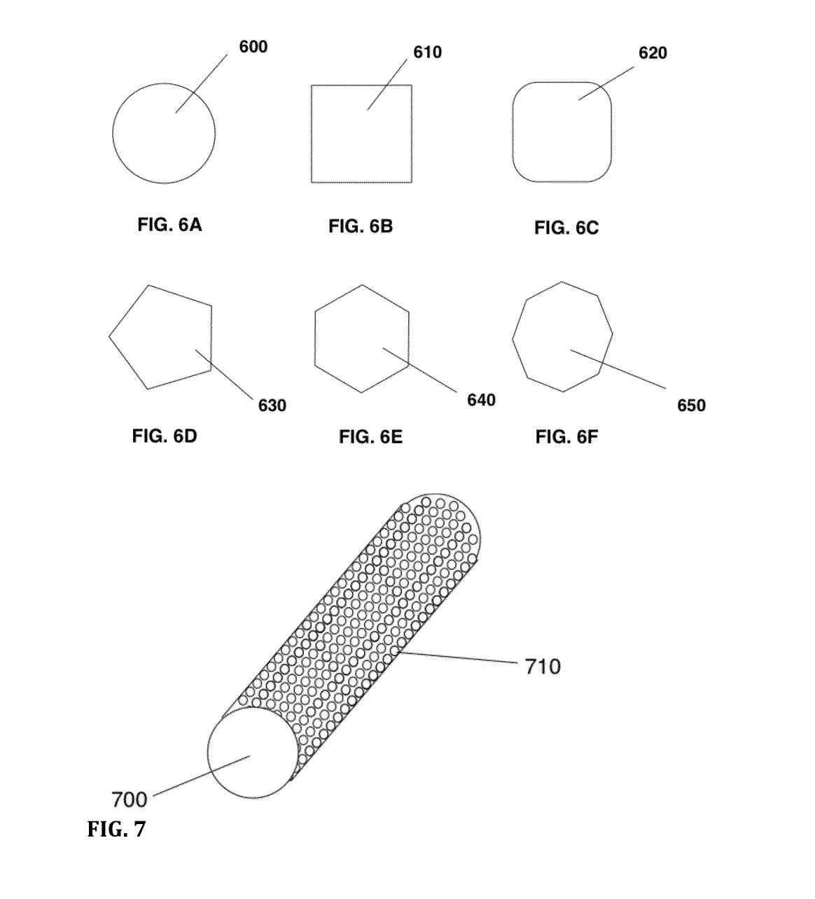

As shown in FIG. 6, Connecting rod 500 in FIG. 5 can be of different cross sections. FIG. 6A illustrates a round cross section 600. FIG. 6B shows a square cross section 610. FIG. 6C demonstrates a rounded-corned cross section 620. FIG. 6D shows a pentagonal cross section. FIG. 6E illustrates a hexagonal cross section 640 and FIG. 6F demonstrates an octagonal cross section 650. Arbitrary cross sections are also part of this invention. An attachment block fastened to a round connecting rod can rotate around the rod unless the fastener is clamped down tightly. If the rod is not round then the clamp cannot rotate because the clamp can move back and forth along the rod, but not around it. The more sides that the shape has, the more resolution there will be in positioning the assembly block around the rod. Thus an octagon can be positioned in twice as many locations as a square. The various embodiments described herein have the advantage of preventing pop-outs of the clamped objects.

FIG. 7 shows a dimpled surface 710 connecting rod or bone pin 700. The surface would match the complementary surface of the associated clamp. A major advantage to the dimpled rod or pin is that the held component is restricted both longitudinally and rotationally. Alternatively the dimple (indent) features on the connecting rods or bone pins could be out-dents. In any case, the features of the connecting rods or bone pins will be opposite and complementary to the features on the surface of an associated clamp. Therefore indent features on the connecting rod or bone pin will engage with out-dent features on the associated clamp. As noted in connection with FIG. 5, Employing such surfaces on connecting rods or bone pins can facilitate use of external fixation assemblies whether quarter-turn clamps are used or not.

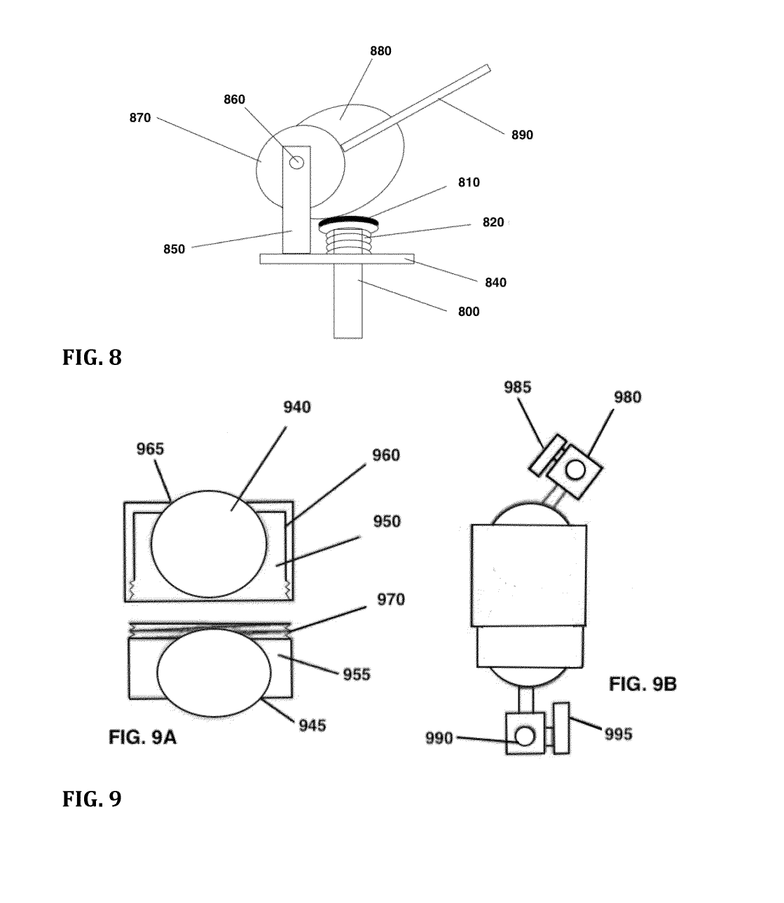

FIG. 8 shows an alternative clamping mechanism, the cam-clamp fastener. The fastener plunger 800 has head 810 and spring 820. Base plate 840 is either attached to the attachment block or incorporated in that attachment block. Base 840 has clamp support 850 with clamp pin 860 providing a pivot point for the cam clamp what consists of small-radius segment 870, large-radius segment 880, and clamp lever 890.

The same approach can be used with a bone pin that equivalently may contained holes bored part of the way through to receive the pin projecting from the end of the quarter-turn fastener.

FIG. 9A shows dual-sphere locking device with quarter-turn clamps: a lockable ball 940 and socket interconnection 960 comprising a ball member 940 and a socket member 950, the socket member defining an entry for the ball member 940, the ball member having two spherical end surfaces, each having a radius of curvature greater than the radius of curvature of the entry 965 in socket member 950, the socket member being adjustable and adapted to receive the ball member such that the ball member is capable of both pivotal and rotational movement within the socket member 950; and lock the ball and socket interconnection such that the ball member and the socket member may be fixed in relative position to each other, the lockable ball and socket interconnection characterized in that an intermediate section is located between the two spherical end surfaces and has a radius of curvature less than the radius of curvature of the entry, and whereby the intermediate section renders the ball member releasable in the socket member. The surface of ball 940 and the matching ball 945 may have dimples as well the openings of socket member 950 and its equivalent 955 in the lower section to engage the balls more securely. This connection is locked by tightening a screw mechanism 970 that can be screwed over an external thread formed on the lower portion of the connecting mechanism. Tightening screw mechanism 970, forces the two ball and sockets against the spherical housing members 950 and 955, locking the balls in a fixed location and angle. As shown in FIG. 9B, clamping to connection rods or bone pins (not shown) is accomplished by rod/pin receptacles 980 and 990 with their respective quarter-turn Fasteners 985 and 995.

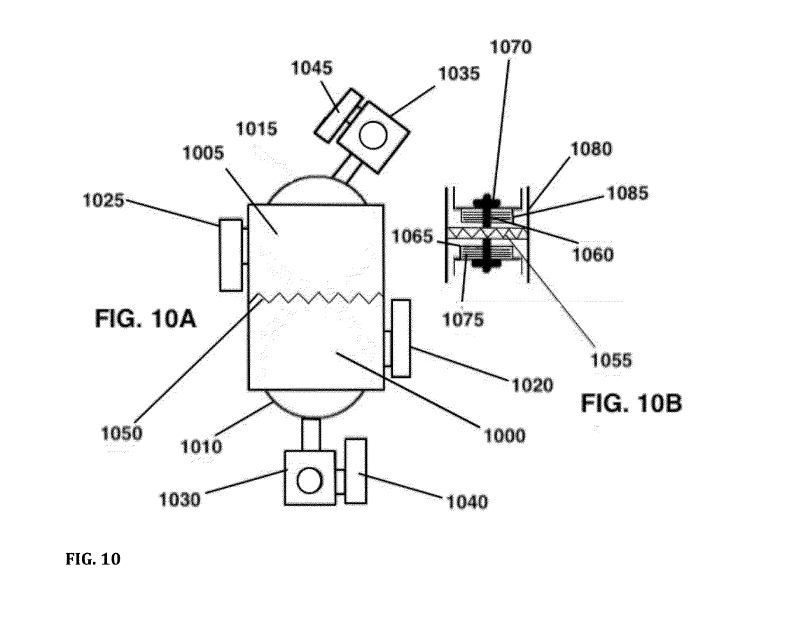

The device in FIG. 10A is a dual-sphere rigid-lock device consisting of lower socket member 1000 and upper lower socket member 1005 with serrated separation interface 1050. Each of the socket members contains movable balls, 1010 and 1015 respectively. The internal structure of the interface that holds the upper and lower members together is shown on the right. As shown in FIG. 10B, a vertical rod with the lower portion of the rod 1065 and upper portion of the rod 1060 with cap 1070 and its equivalent below pierce the serrated interface location 1055 with upper spring 1085 and lower spring 1075 allowing separation of the upper and lower halves by pulling them apart so that the upper and lower halves with their included balls 1015 and 1010 can be rotated relative to each other. When the upper and lower halves are released, they come together and are rotatably fixed. Illustrated in FIG. 10A, quarter-turn or non-quarter turn rod and handle assembly 1020 locks ball 1010 in its position. Quarter turn or non-quarter turn rod and handle assembly 1025 locks Ball 1015 in its position. Connecting rod or bone-pin clamp 1030 affixed to ball 1010 clamps the rod or pin using quarter turn or non-quarter turn rod and assembly 1040. In like manner, connecting rod or bone-pin clamp 1035 affixed to ball 1015 clamps the rod or pin using quarter turn or non-quarter turn rod rod and assembly 1045. FIGS. 9 and 10 are examples of embodiments allowing pivoting and rotating of clamped objects to many different angles.

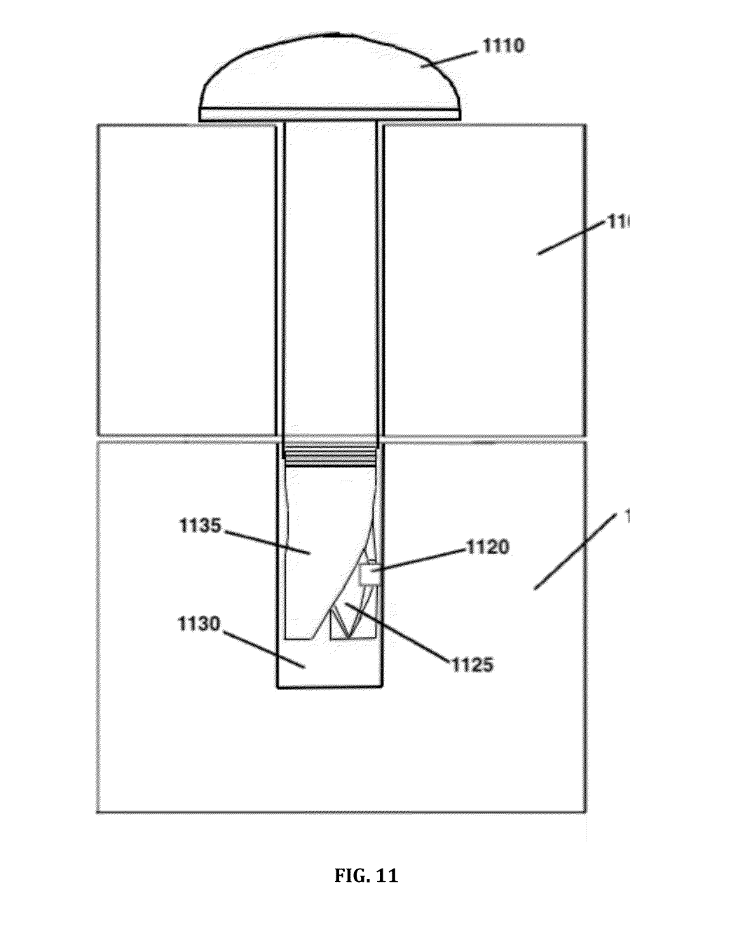

As shown in FIG. 11, a quarter-turn locking mechanism with head 1110 pulling upper housing 1100 and lower housing 1140 together is depicted with the receiving receptacle 1130 that has a protruding pin 1120 that slides within a slot 1125 in the pin shaft 1135 to guide the shaft into locked engagement with the socket. The shaft also has a cantilever body which wedges into a tapered region in the socket to frictionally bind the shaft and socket together. The fixed body is basically composed of the cylinder of the lock, which is connected to a sliding arm, and the arm in turn is connected to a shaft or movable body. When the shaft of the lock is activated, the sliding arm is moved, thus moving the rod's articulator. This small rotational movement of the rod's articulator is sufficient to alter the course of the protruding pin that is associated to the locking elements, locking or unlocking the clamping mechanism. In this embodiment, engaging the quarter-turn fastener draws engaged elements towards head 1110 of the fastener as opposed to the embodiments of FIGS. 1 and 3 in which the shaft is advanced, for example, to be moved into the holes of the rod in FIG. 5.

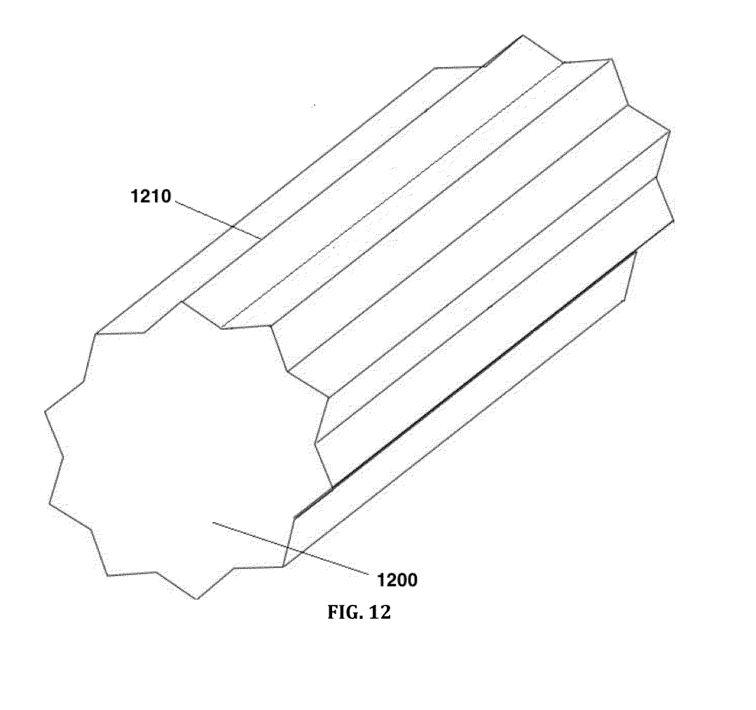

FIG. 12 shows a 10-point connecting rod 1200. As shown, 10-point connecting rod 1200 has 10 triangular features 1210. When the matching clamp, not shown, is simultaneously slipped over the ten triangular features 1210 the matching clamp will not be able to rotate and therefore the relationship between 10-Point Connecting rod 1200 and the matching clamp will be rotationally fixed. General to the field of external fixation, and more specifically, to connection rods having 10 rigid points that allow for both rapid and gradual adjustments and overall strength of the connection of clamps rods. The same principle applies to bone pins. The 10-point connecting rod 1200 is lightweight and simple for fixating bones that is strong enough to hold the bones in their intended positions. The 10 points also allow for more accurate clamping and prevents the connecting rod from “popping out” of the clamp. The number of points not necessarily 10; other number are suitable. The greater the number of points, the more resolution there is to allow finer control of the angular relationship of one pin to another pin.

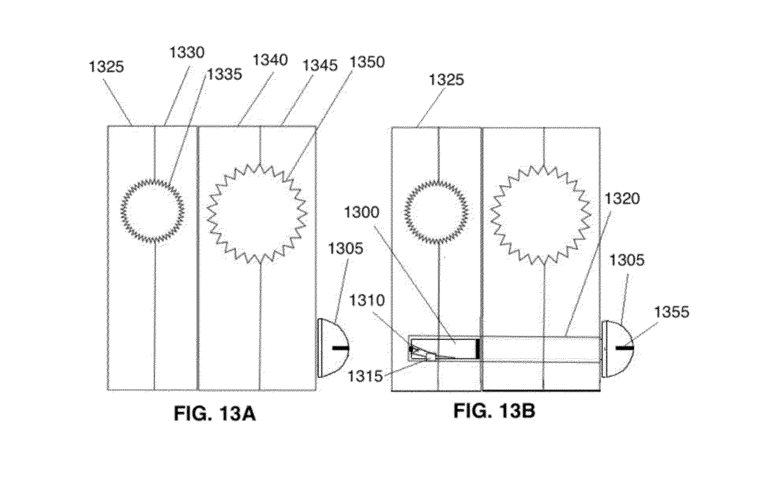

FIG. 13 shows a parallel clamp for external bone fixation. The clamp may include one or more of the following features. For example, the clamping assemblies may be arranged back-to-back with respect to one another. The first-shaped clamping surfaces may be generally parallel, flat faces. The second-shaped clamping surfaces may include non-parallel clamping surfaces, such that two connecting rods or a combination of connecting rods and bone pins may be clamped between the non-parallel clamping surfaces are fixed at a non-parallel angle with respect to each other. A pair of clamps holding a connecting rod, a connecting rod and a bone pin, or two bone pins can be rotated relative to each other around the axis defined by the quarter-turn clamping fixture, the locking mechanism. Types of connections are shown in TABLE 1. Another case is where one of the clamped elements incorporated in the frame system is a support that is only clamped at one end.

TABLE 1

| | CONNECTIONS | | |
|---|---|---|---|
| TYPE | CONNECTION 1 | CONNECTION 2 | CONNECTION 3 |
| Frame | Connecting Rod | Connecting Rod | — |
| Frame | Connecting Rod | Connecting Rod | Connecting Rod |
| Bone Pin | Connecting Rod | Bone Pin | — |
| Bone Pin | Connecting Rod | Bone Pin | |

As shown in FIG. 13A, the parallel clamping jaws may include one clamping jaw set with a star-shape 1350 contained within jaws 1340 and 1345 and another clamping jaw with differently shaped channels 1335 contained within jaws 1325 and 1330. The various styles of channels 1350 and 1335 create a strong and stable base while preventing rotation of the connecting rods and bone pins. This is essential to the foundation of the clamping mechanism. Clamp set pair 1325 and 1130 and clamp set pair 1340 and 1345 may rotate in any angle in respect to each other, where as to meet the optimal bone fixation angle. As shown in FIG. 13B, the clamp assembly is further comprised of a quarter-turn locking mechanism 1305 for locking the parallel clamp sets in a fixed position. The locking occurs with the rotation of shaft 1330 within compartment 1320 with slot 1355 employed to rotate the shaft axis. The quarter-turn locking mechanism works because pin 1315 protruding from compartment 1320 engages in curved slot 1310 in shaft 1300 and as slot 1355 is rotated pulls most distal clamp jaw 1325 thus pulling the remaining clamps and associated connecting rods and bone pins together and locking them all into place. The small rotational movement of the quarter-turn articulator 1305 is sufficient to engage or disengage pin 1315, locking or unlocking the clamping mechanism.

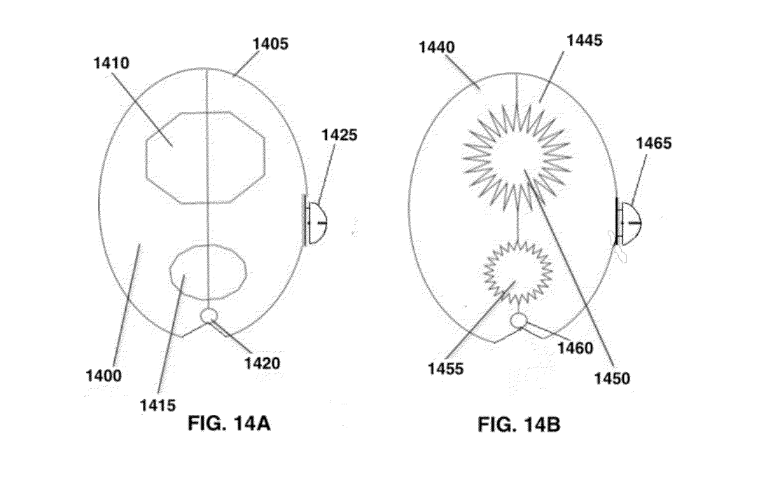

FIG. 14 illustrates parallel clamping devices comprised of jaws 1400 and 1405 in FIG. 14A and jaws 1440 and 1445 in FIG. FIG. 14B. The clamp set made of jaws 1400 and 1405 has eight-sided opening 1410 and 12-sided opening 1415 for holding connecting rods or bone pins has associated pivot hinge 1420. In like manner, the clamp set made of jaws 1440 and 1445 has star-shaped openings 1450 and 1455 for holding connecting rods or bone pins has associated pivot hinge 1460. The non-circular outlines of opening 1410, 1415, 1450, and 1455 demonstrate alternative connecting rod or bone-pin containing shapes that will restrict the clamped elements preventing them from rotating. The respective quarter-turn quick-release latching mechanisms are 1425 in FIG. 14A and 1465 in FIG. 14B.

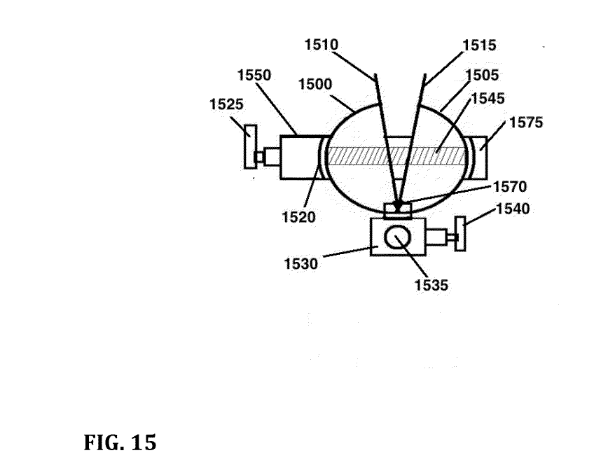

FIG. 15 shows the cross section of a pinch-clamp assembly for engaging connecting rods and bone pins with a locking means in which collapsible sphere with left component 1500 and right component 1505 are rotatably fixed with within bracket 1575 being pushed away from each other by spring 1545. If attached handles 1510 and 1515 are squeezed towards each other, left component 1500 and right component 1505 rotate around hinge pivot 1570 and disengage from bracket 1575 and can rotate right to left, front to back, or a combination of those directions. The outsides of left and right components are covered with dimples and the inside of bracket 1575 is covered with matching divots (or vice versa). When the handles 1510 and 1515 are relaxed, the dimples and divots are engaged and the left and right spherical components locked into place. The hinge pivot 1570 is attached to connecting rod/bone pin clamp 1530. The connecting rod or bone pin are placed within opening 1535 and locked into place with quarter-turn or non-quarter turn locking mechanism 1540. In like manner bracket 1575 has opening 1550 (vertical in FIG. 15 so not visible) with the connecting rod or bone pin able to be locked into place with quarter-turn or non-quarter turn locking mechanism 1525.

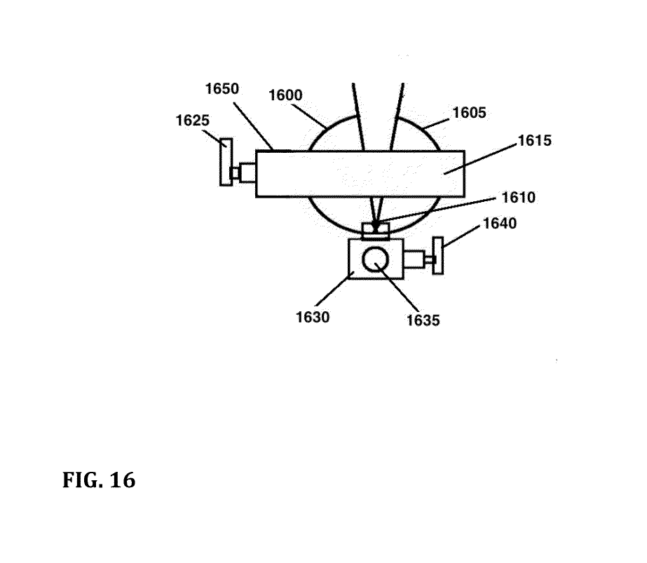

FIG. 16 shows the external view of the pinch-clamp assembly illustrated in FIG. 15. In this view the outside of bracket 1615 that surrounds the collapsible sphere with left component 1600 and right 1605. The articulation of the two components is at hinge pivot point 1610 to which connecting rod/bone pin clamp 1630 with its opening 1635 and locking mechanism 1640. The other connecting rod/bone pin is held in clamp 1650 (vertical in FIG. 16 so not visible) with the connecting rod or bone pin able to be locked into place with quarter-turn or non-quarter turn locking mechanism 1625.

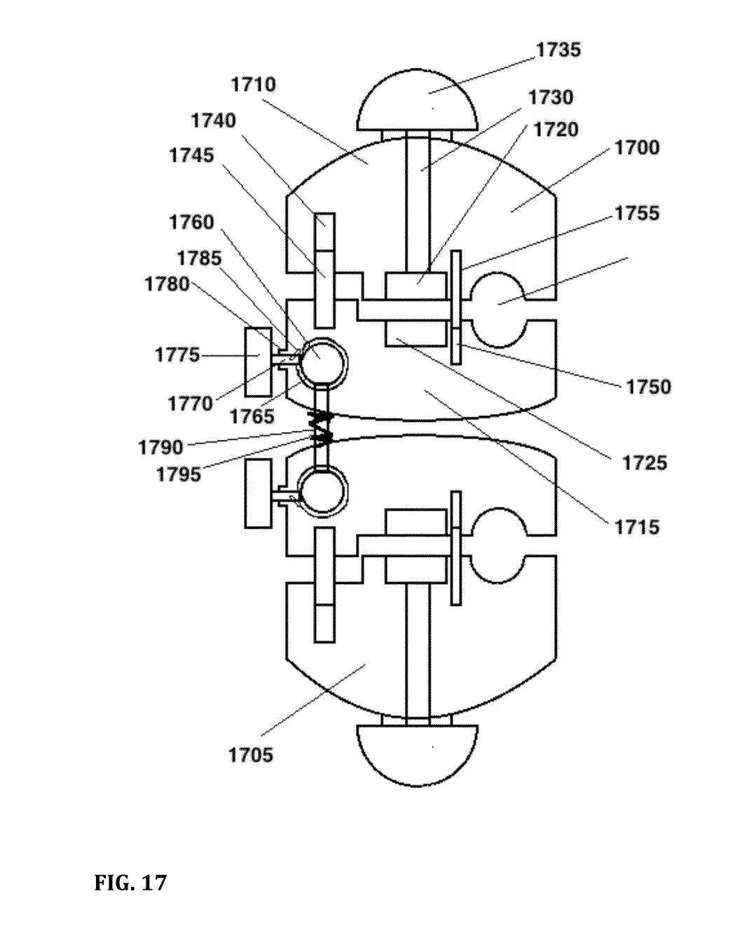

FIG. 17 shows a Quick-Turn, external-fixation clamp with the use of programmable magnets: the figure illustrates an embodiment using programmable magnets showing two clamps, upper clamp 1700 and lower clamp 1705 mechanically interfaced by rod 1790. The details for each of clamps 1700 and 1795 are the same so they are illustrated in upper clamp 1700. This figure shows the Quick Turn clamp utilizing the programmable magnets 1720 and 1725. Programmable magnet 1725 is in a fixed position while programmable magnet 1720 rotates and moves in a vertical position. These magnets can be programmed for greater locking strength and programmed to repel one another at certain rotational orientations so that tools, although they can be used, are not needed. Upper clamp 1700 itself is comprised/composed of upper section 1710 and lower section 1715. Upper section 1710 is kept rotatably fixed relative to lower section 1715 with two slidable elements, the first composed/comprised of compartment 1740 and sliding pin 1745 and the element composed of compartment 1750 and, sliding pin 1755. Programmable magnet 1720 is rotated on shaft 1730 with rotation accomplished by rotating knob 1735. In an alternative embodiment, there can be a shaft protruding from one of the programmable magnet halves 1710 or 1715 and a receiving hole in its opposite half. A spring 1795 surrounding shaft 1790 is used to keep upper and lower clamps in their relative position to each other. At each end of shaft 1790 are ball-and-socket joints located in upper clamp 1700 as ball 1760 contained within compartment 1765. The ball and socket locking mechanism is offset for greater degrees of variance if desired by the user. The ball-and-socket mechanism uses the quick-turn locking mechanism consisting of shaft 1770 enclosed in a hole including boss 1780 that pressed against ball 1760 using quarter-turn catch feature 1785 to secure the clamps in the desired position by turning knob 1775. The twist-release clamp can be used in a variety of extraskeletal-assembly configurations, such as the one shown in FIG. 2.

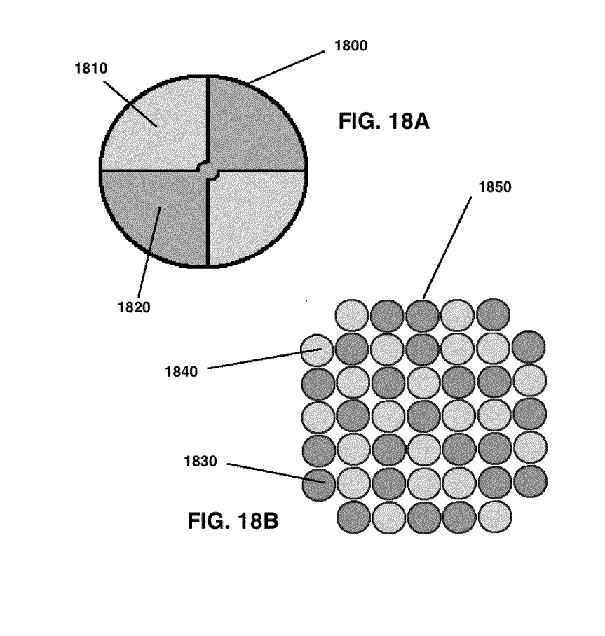

FIG. 18A shows the gross pattern of maxels for a Polymagnet with twist-release behavior. Overall pattern 1800 has two broad fields of maxels 1810 and 1820 with opposite polarities so when the two halves of the Polymagnet (opposite half not shown in figure) are rotated, they will repel as the magnetic regions of the same polarity are exposed to each other and thus the clamp will be released. When one half of the Polymagnet pair is further rotated the magnetic regions will again be of opposite polarity and the clamping will return. The pair of programmable magnets has a strong attractive force when the regions of opposite polarity are facing each other but repel and thus release when the magnet is rotated and regions of opposite polarity are facing each other. During the assembly of the extraskeletal frame, re-clamping would only be applied when a given component of the frame being clamped was in the desired position in three-dimensional space. Twist-release programmable magnets do not have to be set to turn in 90-degree increments; other twist-release angle patterns work as well, such as 45 degrees and 22.5 degrees. The two halves of the magnet must be axially constrained to allow the desired behavior to be exhibited; otherwise the two magnet halves will separate radially. The regions 1810 and 1820 in FIG. 18A are made up of individual maxels. FIG. 18A illustrates a programmable-magnet twist-release pattern of 90 degrees since each of the four sectors has a 90-degree angle. FIG. 18B illustrates a more highly magnified field of maxels 1850. The more darkly shaded maxels represented by maxel 1830 is of opposite polarity of the lightly shaded maxels represented by maxel 1840. Both the rotational torque of the Polymagnets and the attachment force of the Polymagnets are programmed to meet the requirements of the clamping. For extraskeletal fixation, torque will be typically be on the order of 2 to 5 inch-pounds for 90-degree twist-release clamping, although not limited to this range. For example, the rotational-torque ranges in inch-pounds could be in the ranges 0.5 to 3, 3 to 7, 7 to 12, and 12 to 20.

Attachment forces when the clamping is active with the faces of the halves of the programmable magnets is contact can vary from a few pound-forces (lbf) to in excess of 50 lbf according to what is required for the given extraskeletal-fixation frame, for example in the attachment forces in pound-forces are in the ranges of 1 to 10, 10 to 25, 25 to 50, 50 to 100, and 100 to 200.

The programmable magnets, designated as Polymagnets®, are produced on magnetic printers (MagPrinters) from Correlated Magnetics Research (CMR) (U.S. Pat. Nos. 8,648,681, 8,773,268, 9,105,384, and 9,269,482) with the magnets themselves by magnet vendors/service bureaus such as Amazing Magnets, LLC of Anaheim, Calif. and Industrial Magnetics, Inc. of Boyne City, Mich. These vendors also supply translucent magnetic viewing film so the magnetic regions can be displayed. CMR MagPrinters create highly focused, high-intensity magnetic fields that penetrate magnetizable material producing the maxel (magnetic element, like a pixel is a picture element). Each maxel has a specified consistent size, shape, polarity, amplitude, and orientation. The technology is capable of producing high-resolution maxel patterns including overlapping ones. The programmable technology allows production of various behaviors. This patent application employs the “twist-release” behavior. Examples of other behaviors are spring, latch-repel, and rotational alignment.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

We claim the following:

1. A fastening system for fixing clamps or connection blocks to connect two or more clamped objects selected from the group consisting of connecting rod and bone pin to create an assembly for extraskeletal fixation comprising
One or more quick-release fasteners constructed from pairs of programmable magnets that are configured with twist-release behavior where one half of the programmable magnet pair is rotatably fixed and its companion half rotated by a shaft\ and knob for rotating where the pair of programmable magnets have a strong attractive force when the regions of opposite polarity are facing each other but repel and thus release when the magnet is rotated and regions of opposite polarity are facing each other, and tools, although could be used, are not required
to lock both that shaft in place and said clamped object into place when the fastener handle is rotated.

2. The fastening system of claim 1 in which two or more sets of parallel clamps are clamped simultaneously with a single fastener.

3. The fastening system of claim 1 in which the ranges of attachment forces of the programmable magnets in pound-forces are selected from the group consisting of 1 to 10, 10 to 25, 25 to 50, 50 to 100, and 100 to 200.

4. The fastening system of claim 1 in which the ranges of rotational torques of the programmable magnets in inch-pounds are selected from the group consisting of 0.5 to 3, 3 to 7, 7 to 12, and 12 to 20.

5. The fastening system of claim 1 where the programmable-magnet twist-release angle patterns are selected from the group consisting of 90 degrees, 45 degrees, and 22.5 degrees.

6. A clamped object used in the externalskeletal-fixation assemblies of claim 1 the surface of which is non-smooth.

7. The clamped object of claim 6 in which its surface is selected from group consisting of holes, dimples, and out-dents.

8. The clamped object of claim 7 where the surface holes are selected from the group consisting of threaded and non-threaded.

9. The clamped object of claim 6 where the shapes of the surface holes are selected from the group consisting of round, square, rectangular, oval, octagon, hexagonal, octagonal, and arbitrary.

10. A clamped object used in external-skeletal-fixation assemblies of claim 1 that is non-round.

11. The clamped object of claim 10 in which the shape is selected from the group consisting of circular, square, rounded corner, pentagonal, hexagon, octagonal, and arbitrary.

12. A fastening system for fixing clamps or connection blocks to connect two or more clamped objects selected from the group consisting of connecting rod and bone pin to create an assembly for extraskeletal fixation comprising
one or more quarter-turn fasteners each with a shaft attached to handle rotating within a round hole without requiring a tool having a pin riding in a curved track in which the pin is in a location selected from the group consisting of
in the same part of the clamp as the turning handle and thus uses a push motion in which the end of the shaft is pushed against the connection rod or bone pin located in its hole and
in a distal second part of the clamp and thus uses a pull motion in which the second part of the clamp is pulled against the connection rod or bone pin
to lock both that shaft in place and said clamped object into place when the fastener handle is rotated.

13. The fastening system of claim 12 in which two or more sets of parallel clamps are clamped simultaneously with a single fastener.

14. A cam-clamp fastening system not requiring a tool for fixing clamps or connection blocks to connect two or more clamped objects selected from the group consisting of connecting rod and bone pin to create an assembly for extraskeletal fixation comprising
a cylindrical plunger riding in a hole where
the clamped object is contained within a hole in the clamp or connection block with a spring that surrounds the plunger to keep said plunger retracted and where
said plunger is advanced by a cam mechanism where
a base attached to the block in which the hole resides in a way selected from the group consisting of attached to the attachment block and incorporated into that attachment block,
with clamp support allowing rotation of a large-radius segment that rotates via action of a handle attached to a small-radius segment around a clamp pin held in the clamp support such that when the large-radius segment is rotated against the head of the plunger said plunger advances
such that when the plunger is advanced by action of the clamp against a clamped object, said clamped object is locked into place.

15. A clamped object used in external-skeletal-fixation assemblies of claim 14 that is non-smooth.

16. The clamped object of claim 15 in which its surface is selected from group consisting of holes, dimples, and out-dents.

17. The clamped object of claim 16 where the surface holes are selected from the group consisting of threaded and non-threaded.

18. The clamped object of claim 16 where the shapes of the surface holes are selected from the group consisting of round, square, rectangular, oval, octagon, hexagonal, octagonal, and arbitrary.

19. A clamped object used in external-skeletal-fixation assemblies of claim 14 that is non-round.

20. The clamped object of claim 19 in which the shape is selected from the group consisting of circular, square, rounded corner, pentagonal, hexagon, octagonal, and arbitrary.
